(19) United States
(12) Patent Application Publication
(10) Pub. No.: US 2004/0199225 A1
(43) Pub. Date: Oct. 7, 2004

(54) Body tanning decal

(76) Inventor: Kathy Patterson, Sharpsburg, GA (US)

Correspondence Address: GARDNER GROFF, P.C., PAPER MILL VILLAGE, BUILDING 23, 600 VILLAGE TRACE, SUITE 300, MARIETTA, GA, 30067, US

(21) Appl. No.: 10/405,687

(22) Filed: Apr. 2, 2003

(51) Int. Cl. A61N 1/00

(52) U.S. Cl. 607/91

(57) ABSTRACT

A decal for tanning a body part over the course of a series of tanning sessions. The tanning decal has a first transparent sheet and a second opaque sheet having a design. In a first exemplary embodiment, the second sheet is removably adhered to the first sheet so that the first sheet can be removed from the second sheet for tanning the design on the body part. In a second exemplary embodiment, the second sheet is adhered to or formed into the first sheet so that it can be left on the body part for tanning the design on the body part. Methods for tanning the body part using the first and second exemplary embodiments are also provided.

### TECHNICAL FIELD

The present invention relates generally to body tanning decals and, more particularly, to body tanning decals for use over the course of a series of tanning sessions.

### BACKGROUND OF THE INVENTION

Many people tan their bodies by natural sunlight at the beach, a deck at their homes, or elsewhere, or by artificial light in tanning beds or booths at home or in tanning salons. Sometimes these people use tanning decals to form a design on their bodies that is fun, affectionate, motivational, interesting, etc. Common designs include hearts, bunnies, and other solid shapes. Such tanning decals typically consist of an adhesive-backed sheet that can be positioned on the body part to be tanned and then removed after the tanning session.

To avoid skin damage from overexposure, most people tan these designs onto their bodies over a series of tanning sessions to cumulatively obtain the desired contrast and tanning effect. But repositioning a new decal precisely over a spot previously tanned can be difficult with conventional tanning decals. For example, when repositioning a subsequent decal in precise alignment over a previously tanned area, it is more difficult when the decal is star-shaped than when it is circular-shaped. So sometimes the decal is not precisely aligned and then the resulting tanned design is not as well-defined and distinct as is desired.

Furthermore, known tanning decals are limited to designs with solid shapes such as those mentioned. There are no known tanning decals with designs that are words or phrases. This is because a decal in the shape of a name written in cursive would be nearly impossible to handle, align, and hold steady when repositioning over a previously tanned area. And such a decal would stretch and/or tear when trying to align it over the previously tanned area. As a result, it would be almost impossible to obtain a distinct and well-defined tanned design of the name. In addition, the damaged decal could not be reused for further tanning.

Accordingly, it can be seen that a need remains for a tanning decal that can be easily and precisely aligned over a design previously tanned onto the user's body, permitting the user to tan a distinct and well-defined design onto his or her body. In addition, there is needed such a tanning decal for tanning a name, word, phrase, or complicated symbol onto the user's body. Furthermore, a need exists for such a tanning decal that can be used once or reused over a series of tanning sessions. It is to the provision of a tanning decal meeting these and other needs that the present invention is primarily directed.

### SUMMARY OF THE INVENTION

Generally described, the present invention provides an innovative tanning decal for tanning a design onto a body part during one or over a series of tanning sessions. The tanning decal has a first sheet for permitting the decal to be properly positioned on the user's body and a second sheet removably adhered to or integrally formed with the first sheet. The second sheet is opaque and has the shape of a design to be tanned onto the user's body. The design can be a heart, bunny, or other solid shape similar to conventional tanning decal designs. Or, unlike conventional tanning decals, the design can be a name, other word, phrase, or combination of words and shapes.

In a first exemplary embodiment, the second sheet of the tanning decal has a top side removably adhered to a bottom side of the first sheet, and the second sheet is removably adherable to the body part. The first sheet can be removed from the second sheet, which stays on the body part for tanning the design. This is what permits using second sheet designs having the shape of a name, other word, phrase, or combination of words and shapes. Preferably, the first sheet is generally rectangular and has a protruding portion for grasping to remove it from the second sheet. In addition, the first sheet preferably has at least a portion that is generally transparent for aligning the second sheet design with the tanned design from a previous tanning session. This permits the decals to be used over a series of tanning sessions to progressively tan a well-defined design onto the user's body. Alternatively, the first sheet can be translucent or opaque, which arrangement is suitable for use during only one tanning session.

This and similar embodiments can be used in a first exemplary method for tanning a body part. In the method, the decal is selectively positioned on the body part, the second sheet adhered to the body part, the first sheet removed from the second sheet without damaging the second sheet, the body part tanned to form thereon the second sheet design, and the second sheet removed from the body part. Then in a later tanning session, the same or a similar decal can be positioned on the body part with its second sheet design aligned with the tanned design on the body part by viewing through the generally transparent portion of the first sheet. In this way, the design can have the shape of most any design desired, including names and other words.

In a second exemplary embodiment, the first sheet is generally transparent for aligning the second sheet design with the tanned design from a previous tanning session, and the second sheet is adhered to or formed into or onto the first sheet. For example, the second sheet can be marked or adhered onto either a top side or a bottom side of the first sheet. Or the second sheet can be integrally formed into the first sheet. And either the first sheet or the second sheet has at least a portion that is removably adherable to the body part. In any of these configurations, the first sheet and the second sheet stay on the body part for tanning the design, with the lights rays passing through the generally transparent first sheet. This is what permits using second sheet designs having the shape of a name, other word, phrase, or combination of words and shapes. Preferably, the first sheet is generally rectangular and has a protruding portion for grasping to remove it from the body part without damaging the second sheet. This and the generally transparent first sheet permit the decal to be used over a series of tanning sessions to progressively tan a well-defined design onto the user's body.

This and similar embodiments can be used in a second exemplary method for tanning a body part. In the method, the decal is selectively positioned on the body part, the first sheet or the second sheet adhered to the body part, the body part tanned through the generally transparent first sheet to form thereon the second sheet design, the decal removed from the body part, and the same or a subsequent decal positioned on the body part with its second sheet aligned with the tanned design on the body part by viewing through the generally transparent portion of its first sheet. In this way, the design can have the shape of most any design desired, including names and other words.

Accordingly, the unique features of the tanning decal of the present invention permit the user to apply one of the decals for one tanning session, remove it afterward, and reapply another one of the decals with the same design over precisely the same spot. This configuration enables the use of designs more irregular and complex than simple solid shapes, so that names, other words, phrases, and combinations of words and symbols can be tanned onto the user's body. In this way, over a series of sessions the user can easily obtain a distinct depiction of the design with the desired contrast between the tanned part and the surrounding parts of the user's body. Or the user can apply one of the decals for a single use to obtain the desired tanning effect.

These and other features and advantages of the present invention will become more apparent upon reading the following description in conjunction with the accompanying drawing figures.

### BRIEF DESCRIPTION OF THE DRAWING FIGURES

FIG. 1 is a plan view of a body tanning decal according to a first exemplary embodiment of the present invention, showing a first sheet and a second sheet having a design.

FIG. 2 is an elevation view of the body tanning decal of FIG. 1.

FIG. 3 is an elevation view of the body tanning decal of FIG. 1, showing the decal selectively positioned on the body part

FIG. 4 is an elevation view of the body tanning decal of FIG. 3, showing the first sheet being removed from the second sheet.

FIG. 5 is an elevation view of the body tanning decal of FIG. 3, showing the body part being tanned to form thereon the second sheet design.

FIG. 6 is an elevation view of the body tanning decal of FIG. 3, showing the second sheet being removed from the body part.

FIG. 7 shows a subsequent decal like that of FIG. 3 being positioned on the body part with its second sheet being aligned with the tanned design on the body part by viewing through the generally transparent portion of its first sheet.

FIG. 8 is a plan view of a body tanning decal according to a second exemplary embodiment of the present invention, showing a first sheet and a second sheet having a design.

FIG. 9 is an elevation view of the body tanning decal of FIG. 8, showing the second sheet adhered onto the top of the first sheet.

FIG. 9A is an elevation view of the a first alternative embodiment of the body tanning decal of FIG. 8, showing the second sheet adhered to the bottom of the first sheet.

FIG. 9B is an elevation view of the body tanning decal of FIG. 8, showing the second sheet formed into the first sheet.

### DETAILED DESCRIPTION OF THE EXEMPLARY EMBODIMENTS

Referring now to the drawing figures, wherein like reference numerals represent like parts throughout the several views, the present invention provides a tanning decal for tanning a design onto a body part during one or over a series of tanning sessions. The decal can be used to tan the design on an arm, a leg, a normally concealed body part, or elsewhere on the human body. The unique features of the decal permit the user to apply one of the decals for one tanning session, remove it afterward, and reapply another one of the decals with the same design over the same spot. In this way, over a series of sessions the user can easily obtain a distinct depiction of the design with the desired contrast between the tanned part and the surrounding parts of the user's body.

The design can be a name, other word, phrase, symbol, or combination thereof. For example, the design can be the name of a husband, wife, child, famous person, or other person. Also, the design can be a phrase such as a teasing or famous slogan. Or the design can be a symbol such as a heart, bunny, unicorn, flower, or lightning bolt, or it can be a combination of symbols and words. It will be understood that the design can be of most anything that users may wish to tan onto their bodies.

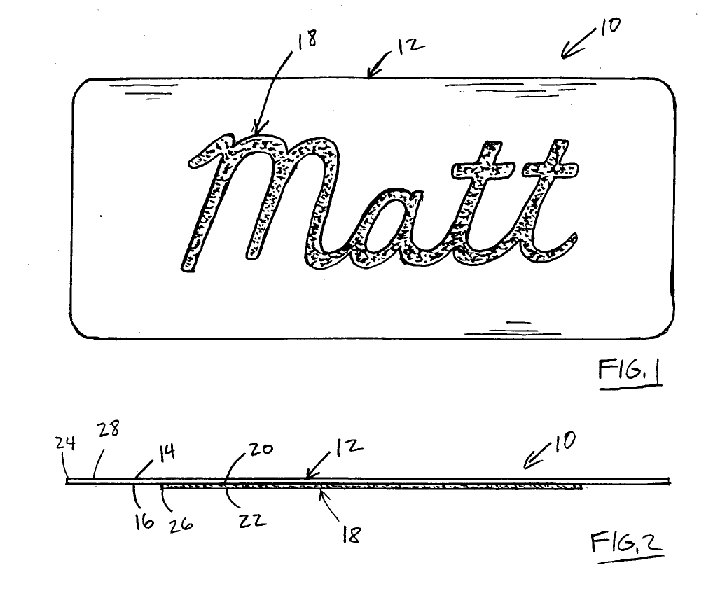

FIGS. 1 and 2 show a first exemplary embodiment of the present invention, referred to generally as the tanning decal 10. The tanning decal 10 includes a first sheet 12 with a top side 14 and a bottom side 16. The first sheet 12 is made of a material that is generally transparent, or at least has a portion immediately surrounding the design that is, so that the user can see a previously tanned design on the body through the first sheet to align the design for additional tanning. For example, in a typical commercial embodiment the first sheet is made of a flexible clear plastic with a generally rectangular shape.

In addition, the tanning decal 10 has a second sheet 18 with a top side 20 and a bottom side 22. The second sheet 18 has the shape of the design to be tanned on the user's body, such as the name “MATT” that is shown in the drawing figure. As such, the second sheet 18 is made of a material that is generally opaque so that it blocks natural and/or artificial lights rays from passing through it. For example, in a typical commercial embodiment the second sheet is made of a flexible plastic with ink, dye, or another pigment formed into or marked onto it.

The top side 20 of the second sheet 18 is adhered to the bottom side 16 of the first sheet 12 so that the first sheet can be selectively removed from the second sheet 18 when desired. And the bottom side 22 of the second sheet 18 has an adherent so that the second sheet can be selectively removed from the body when desired. For example, the adherent between the first and second sheets 12 and 18 and that on the second sheet bottom 22 can be a conventional type that is commonly used on known body decals. In this way, the bottom side 22 of the second sheet 18 can be adhered to the body part and then the first sheet 12 can be removed from the second sheet for tanning the design on the user's body. Then the second sheet 18 can be removed from the user's body and later another decal 10 can be applied over precisely the same location of the body for further tanning.

To make it easy to remove the first sheet 12 from the second sheet 18, in a typical commercial embodiment the first sheet has a periphery 24 and the second sheet has a periphery 26 that is smaller that the first sheet periphery. In this way, the first sheet 12 has at least one portion 28 that protrudes beyond the second sheet periphery 24 for grasping to peel back or otherwise remove the first sheet from the second sheet. Preferably, but not necessarily, this protruding portion 28 does not have the adherent on it so that it can be easily pulled back from the body by the user's fingernails.

The tanning decal 10 can be manufactured by conventional fabrication techniques known in the art. Also, an array of the decals 10 can be made on a single sheet, which can be sold in a folded, rolled, or other arrangement.

In an alternative embodiment for single-tanning-session applications, the first sheet is made of a material that is generally opaque. In this form the decal can be used once or multiple times, although it is not as easily repositioned over a previously tanned design.

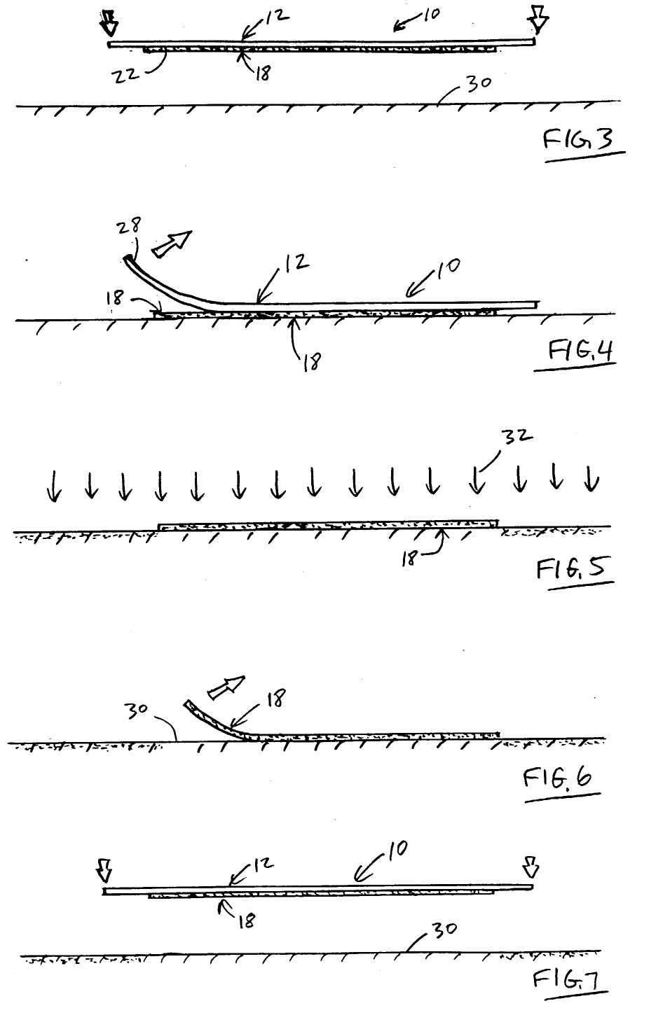

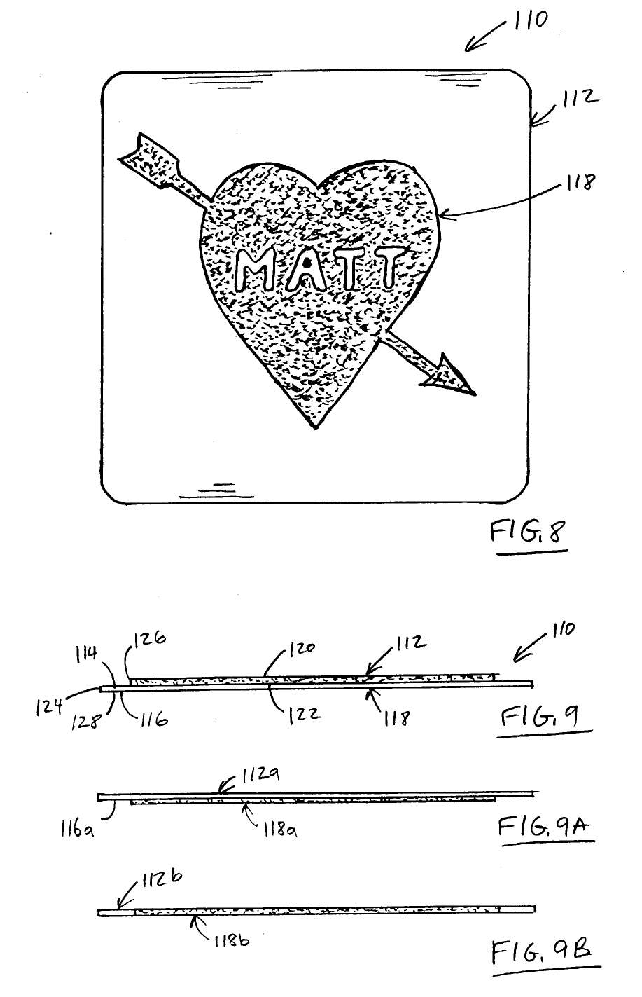

FIGS. 3-7 show a first exemplary method for tanning a design on a body part using the tanning decal 10. FIG. 3 shows the decal 10 being selectively positioned on the surface 30 of the body part to be tanned. The bottom 22 of the second sheet 18 is then adhered to the body part surface 30 in the selected position. FIG. 4 shows the first sheet protruding portion 28 being peeled back to remove the first sheet 12 from the second sheet 18 while the second sheet remains adhered to the body part. FIG. 5 shows the second sheet 18 adhered in position and the body part being tanned by natural or artificial light rays 32 to form the design of the second sheet on the body part. FIG. 6 shows the second sheet 18 being peeled back to remove it from the body part, leaving a contrast between the untanned surface 30 of the body and the immediately surrounding tanned area. This contrast forms the desired design. FIG. 7 shows another decal 10 with its second sheet 18 having the same design being positioned on the body part so that the second sheet is aligned with the untanned surface design 30 of the body part. The second sheet 18 of the decal 10 can be aligned with the tanned design by looking through the generally transparent portion of the first sheet 12 for further tanning to produce a well-defined depiction of the design with additional contrast.

Alternatively, to remove the second sheet 18 from the body, the first sheet 12 can be pressed down onto the second sheet and re-adhered to it. Then the first sheet 12 can be used to remove the second sheet from the body. Then the same decal can be reused for subsequent tanning sessions instead of using a new decal.

FIGS. 8 and 9 show a second exemplary embodiment of the present invention, referred to generally as the tanning decal 110. Similarly to the first embodiment, the tanning decal 110 has a first sheet 112 with a top side 114 and a bottom side 116 and that is made of a material that is generally transparent, or at least has a portion immediately surrounding the design that is generally transparent. And the tanning decal 110 has a second sheet 118 with a top side 120 and a bottom side 122 and that has the shape of the design to be tanned on the user's body, such as the heart-shaped symbol with the name “MATT” that is shown in the drawing figure.

In a typical commercial embodiment shown in FIG. 9, the second sheet 118 is adhered or marked onto the top side 114 of the first sheet 112, with the second sheet preferably raised only slightly above the first sheet. In an alternative embodiment shown in FIG. 9A, the second sheet 118a is similarly adhered or marked onto the bottom side 116a of the first sheet 112a. And in another an alternative embodiment shown in FIG. 9B, the second sheet 118b is integrally formed into the first sheet 112b. In any event, decal 110 can be adhered to the body part and left there during tanning, then afterward removed and discarded or reused.

To make it easy to remove the first sheet 112 from the second sheet 118 without accidentally picking at and thus damaging the second sheet, in a typical commercial embodiment the first sheet has a periphery 124 and the second sheet has a periphery 126 that is smaller that the first sheet periphery. In this way, the first sheet 112 has at least one portion 128 that protrudes beyond the second sheet periphery 124 for grasping to peel back or otherwise remove the first sheet from the second sheet without touching the second sheet.

In a second exemplary method for tanning a design on a body part, the tanning decal 110 is selectively positioned on the body part to be tanned (similarly to FIG. 3). Using this method, either the first or the second sheet of the decal 110 is adhered to the body part, depending on which of the aforementioned second embodiments is used (see FIGS. 9, 9A, and 9B). Then the body part is tanned through the first sheet by natural or artificial light rays to form the design of the second sheet on the body part (similar to FIG. 5 but with the first sheet intact). Next the decal 110 is removed from the body part, leaving a contrast between the untanned surface of the body and the immediately surrounding tanned area. This contrast forms the desired design. And then the same or another decal 110 with its second sheet having the same design is positioned on the body part so that the second sheet is aligned with the untanned surface design of the body part. The second sheet of the decal can be aligned with the tanned design by looking through the generally transparent portion of the first sheet for further tanning to produce a well-defined depiction of the design with additional contrast.

Accordingly, the present invention provides a tanning decal with innovative features that permit the user to apply one of the decals for one tanning session, remove it afterward, and reapply another one of the decals with the same design over precisely the same spot. This configuration enables the use of designs more irregular and complex than simple solid shapes, so that names, other words, phrases, and combinations of words and symbols can be tanned onto the user's body. In this way, over a series of sessions the user can easily obtain a distinct depiction of the design with the desired contrast between the tanned part and the surrounding parts of the user's body. Or the user can apply one of the decals for a single use to obtain the desired tanning effect.

In the embodiments described above and in the following claims, the words “a,” “an,” and “one” are not intended to mean “only one” but can also mean any number greater than one. Similarly, plural terms are sometimes used for convenience and are not necessarily intended to mean “more than one” but can also mean “only one.” Additionally, the methods are not intended to be limited to the particular sequence of steps described.

While the invention has been shown and described in exemplary forms, it will be apparent to those skilled in the art that many modifications, additions, and deletions can be made therein without departing from the spirit and scope of the invention as set forth in the following claims.

What is claimed is:

1. A tanning decal for a body part, comprising:
a first sheet having a bottom side; and
a second sheet having a top side and a bottom side, the top side removably adhered to the first sheet bottom side and the bottom side removably adherable to the body part, wherein the second sheet is generally opaque and has the shape of a design,
wherein the decal may be selectively positioned on the body part, the second sheet adhered to the body part, the first sheet removed from the second sheet without damaging the second sheet, the body part tanned to form thereon the second sheet design, and the second sheet removed from the body part.

2. The tanning decal of claim 1, wherein the second sheet design comprises a name, other word, phrase, symbol, or combination thereof.

3. The tanning decal of claim 1, wherein the first sheet has a periphery and the second sheet has a periphery that is smaller that the first sheet periphery.

4. The tanning decal of claim 3, wherein the first sheet has a protruding portion for grasping to remove the first sheet from the second sheet.

5. The tanning decal of claim 1, wherein the first sheet has at least a portion that is generally transparent so that the same or a subsequent decal can be positioned on the body part with its second sheet aligned with the tanned design on the body part by viewing through the generally transparent portion of its first sheet.

6. The tanning decal of claim 5, wherein the first sheet has a periphery, the second sheet has a periphery that is smaller that the first sheet periphery, and the first sheet has a protruding portion for grasping to remove the first sheet from the second sheet.

7. The tanning decal of claim 1, wherein the first sheet is generally rectangular.

8. The tanning decal of claim 1, wherein the first sheet and the second sheet are made of a plastic material.

9. A tanning method, comprising:
providing a tanning decal according to claim 5;
selectively positioning the tanning decal on the body part;
adhering the second sheet to the body part;
removing the first sheet from the second sheet;
tanning the body part to form thereon the second sheet design;
removing the second sheet from the body part; and
positioning on the body part the same or a subsequent decal with its second sheet aligned with the tanned design on the body part by viewing through the generally transparent portion of its first sheet.

10. A tanning decal for a body part, comprising:
a first sheet that is generally transparent; and
a second sheet that is generally opaque, has the shape of a design, and is formed onto or into the first sheet, the first sheet or the second sheet having at least a portion that is removably adherable to the body part,
wherein the decal may be selectively positioned on the body part, the first sheet or the second sheet adhered to the body part, the body part tanned through the generally transparent first sheet to form thereon the second sheet design, the decal removed from the body part, and the same or a subsequent decal positioned on the body part with its second sheet aligned with the tanned design on the body part by viewing through the generally transparent portion of its first sheet.

11. The tanning decal of claim 10, wherein the second sheet is marked onto or adhered to a top side or a bottom side of the first sheet.

12. The tanning decal of claim 10, wherein the second sheet is integrally formed into the first sheet.

13. The tanning decal of claim 10, wherein the second sheet design comprises a name, other word, phrase, symbol, or combination thereof.

14. The tanning decal of claim 10, wherein the first sheet has a periphery, the second sheet has a periphery that is smaller that the first sheet periphery, and the first sheet has a protruding portion for grasping to remove the decal from the body part without damaging the second sheet.

15. The tanning decal of claim 10, wherein the first sheet is generally rectangular.

16. The tanning decal of claim 10, wherein the first sheet and the second sheet are made of a plastic material.

17. A tanning method, comprising:
providing a tanning decal according to claim 9;
selectively positioning the tanning decal on the body part;
adhering the first sheet or the second sheet to the body part;
tanning the body part through the first sheet to form thereon the second sheet design;
removing the tanning decal from the body part; and
positioning on the body part the same or a subsequent decal with its second sheet aligned with the tanned design on the body part by viewing through the generally transparent portion of its first sheet.
